(19) United States
(12) Patent Application Publication
(10) Pub. No.: US 2021/0052485 A1
(43) Pub. Date: Feb. 25, 2021

(54) COSMETIC COMPOSITION AND PREPARATION METHOD

(71) Applicant: C.G.S. S.R.L., Monza (IT)

(72) Inventor: Cecilia Garofano, Monza (IT)

(21) Appl. No.: 17/090,837

(22) Filed: Nov. 5, 2020

Related U.S. Application Data
(63) Continuation of application No. 16/085,271, filed on Sep. 14, 2018, now abandoned.

(30) Foreign Application Priority Data
Mar. 14, 2016 (IT) 102016000026298

(51) Int. Cl. A61K 8/9789; A61K 8/97; A61Q 19/08; A61K 36/82; A23F 3/18; A61Q 19/00

(52) U.S. Cl. CPC A61K 8/9789; A61K 8/97; A61Q 19/08; A61K 36/82; A61K 2800/805; A61Q 19/00; A61K 2236/331; A61K 2800/522; A23F 3/18

(57) ABSTRACT

Cosmetic composition, with no added aqueous phase and comprising a tea infusion to replace the aqueous phase, combined with at least a composition component for personal care.

This application is a continuation of U.S. application Ser. No. 16/085,271 filed on Sep. 14, 2018 and published as U.S. Patent Publication No. 2019/0070098. U.S. application Ser. No. 16/085,271 is a national stage of PCT/EP2017/055924 filed on Mar. 14, 2017, which itself claims priority to Italian Application No. IT102016000026298 filed on Mar. 14, 2016. Each of the above-mentioned applications are expressly incorporated herein by reference.

### FIELD OF THE INVENTION

Embodiments described here concern a cosmetic composition, in particular based on an infusion of tea, and a method for preparing said cosmetic composition.

### BACKGROUND OF THE INVENTION

As the population gradually becomes aware of the potentially adverse effects on the body of external pollutant agents, factors that age the organism, anti-esthetic factors connected to the skin, hair, nails etc., the cosmetics and personal care industry has increased its search for natural ingredients that bring benefits to the organism thanks to their intrinsic properties and the compounds or active principles that are naturally present therein, such as anti-oxidant, nutrient and anti-ageing properties and suchlike.

In this context, therefore, there is a growing need to make available cosmetic compositions, for example in the form of creams, lotions or other, which provide active principles or compounds that are useful for the organism, that are natural in origin and that maintain unchanged, or at least at a satisfactory level, also in the final product, all their intrinsic beneficial properties for the organism, the skin, hair, nails, etc.

However, this need cannot always be satisfied efficiently or easily, since the techniques for preparing the cosmetic compositions in question, or the ingredients that are inserted in their formulas, can be detrimental to maintaining the beneficial properties of these natural compounds or active principles, whose beneficial properties are sought. Indeed it is known that for preparing the cosmetic compositions, for example those that are spread, such as creams, or mask or packs, water is typically used, in particular using the water emulsion technique (oil in water or water in oil), and a source of energy, since the preparation is typically made hot, that is, at a temperature that can vary from 70° C. to 80° C. These operating and process conditions can damage or render ineffective the beneficial compounds in question, reducing or eliminating the beneficial effects connected with natural compounds or active principles present in the formulas.

Other limitations and disadvantages of conventional solutions and technologies will be clear to a person of skill after reading the remaining part of the present description with reference to the description of the embodiments that follow, although it is clear that the description of the state of the art connected to the present description must not be considered an admission that what is described here is already known from the state of the prior art.

There is therefore a need to perfect a cosmetic composition and a method to prepare it that can overcome at least one of the disadvantages of the state of the art.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

According to some embodiments a cosmetic composition is provided. According to one embodiment, the cosmetic composition has no added aqueous phase and comprises a tea infusion to replace the aqueous phase, combined with at least one composition component for personal care.

According to possible embodiments, the infusion of tea is an infusion of any type of tea coming from the Camellia sinensis plant. Although this does not restrict the field of protection of the invention, the infusion can for example be an infusion of one or more teas chosen from a group consisting of: white tea, black tea, green tea, blue tea (Oolong) and red tea.

According to possible embodiments, the green tea can be, for example, of the Matcha variety.

According to possible embodiments, the cosmetic composition is for topical application on the skin, and/or hair and/or nails.

According to possible variant embodiments, the cosmetic composition is a cosmetic product chosen from cream, in particular a cream for skin care, a body cream, a face cream, a hand cream, a leg cream, a foot cream, a hair cream, a soothing balsam cream for the hair, a cream for around the eyes or for the eyelids, or an anti-cellulite, slimming or remodeling cream or gel, an exfoliator product for the face or body, a make-up removing lotion, UVA-UVB protection creams or lotions, tanning creams and lotions, perfumes for the body, make-up products such as foundation cream, powder or compacts, lipsticks or lip balms, tanning powders, or a composition for personal cleanliness, in particular a shower gel, shampoo, bubble bath, soap for personal cleanliness, or face mask composition, or a lotion, a cosmetic serum, a serum in drops, cosmetic mud pack, cosmetic gel.

According to another aspect of the present description, some embodiments provide the use of a cosmetic composition, with no added aqueous phase and comprising a tea infusion to replace the aqueous phase, combined with at least one composition component for personal care, for topical application on the skin, and/or hair and/or nails.

According to yet another aspect of the present description, some embodiments provide a method for preparing a tea infusion for use in the preparation of a cosmetic composition, with no added aqueous phase and comprising said tea infusion to replace the aqueous phase, combined with at least one composition component for personal care. According to one embodiment, the method comprises:
making available hot water;
adding tea leaves to the hot water;
infusing the tea leaves in the hot water;
filtering the tea infusion.

According to possible embodiments, the method for the preparation of a tea infusion comprises:
weighing and loading the water in a turbo-emulsifier and bringing it to the boil by activating a heating system of the turbo-emulsifier;
switching off the heating system to make the temperature of the water go down to a temperature correlated to the type of tea leaves used for the infusion;
adding the tea leaves to the water in the turbo-emulsifier;
mixing the tea leaves and water in the turbo-emulsifier;
stopping the mixing;
letting the infusion of tea leaves continue in the hot water in the turbo-emulsifier for a predefined infusion time;
cooling the tea infusion obtained by rapidly bringing down the temperature in the turbo-emulsifier;
filtering and packing the tea infusion.

According to another aspect of the present description, some embodiments provide a method to prepare a cosmetic composition in emulsion and with no added aqueous phase, said method providing to use a tea infusion to replace the aqueous phase prepared according to a method for the preparation of a tea infusion according to the present description and the combination of the tea infusion with at least a cosmetic composition for personal care.

These and other aspects, characteristics and advantages of the present disclosure will be better understood with reference to the following description, drawings and attached claims. The description is intended to describe the principles of the disclosure.

The various aspects and characteristics described in the present description can be applied individually where possible. These individual aspects, for example aspects and characteristics described in the attached dependent claims, can be the object of divisional applications.

It is understood that any aspect or characteristic that is discovered, during the patenting process, to be already known, shall not be claimed and shall be the object of a disclaimer.

### DETAILED DESCRIPTION OF SOME EMBODIMENTS

We shall now refer in detail to the various embodiments of the present invention, of which one or more examples are shown hereafter. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

All the measurements are carried out, unless otherwise indicated, at 25° C. and atmospheric pressure. All the temperatures, unless otherwise indicated, are expressed in degrees Celsius.

All the percentages and ratios indicated refer to the weight of the total cosmetic composition (w/w), unless otherwise indicated.

All the percentage intervals reported here are supplied with the provision that the sum with respect to the overall cosmetic composition is 100%, unless otherwise indicated.

All the intervals reported here shall be understood to include the extremes, including those that report an interval “between” two values, unless otherwise indicated.

The present description also includes the intervals that derive from uniting or overlapping two or more intervals described, unless otherwise indicated.

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

Terms such as “about”, “generally”, “substantially” and suchlike shall be understood with their function of modifying a term or value that is not absolute, but is not reported in the state of the art. Such terms shall be defined by the specific circumstances and by the terms that they are intended to modify according to the common acceptance of such terms in the specific field. They shall take into account at least the degree of experimental error expected, the technical error and the instrumental error for a given technique adopted to measure a value. Unless otherwise indicated, in the present description, singular forms such as “a”, “an” and “one” shall be understood to include plural forms, unless the context suggests otherwise.

Embodiments described here concern a cosmetic composition with no added aqueous phase and comprising an infusion of tea to replace the aqueous phase, combined with at least one composition component for personal care.

Embodiments described here provide that the cosmetic composition described here is for topical application on the skin, hair and/or nails.

Other embodiments described here concern a method for preparing a cosmetic composition with no added aqueous phase, where an infusion of tea is used to replace the aqueous phase, combined with at least one composition component for personal care.

By the expression tea, here and in the present description, we mean Camellia sinensis. The infusion of tea is understood to be made with tea leaves (Camellia sinensis leaves).

The composition according to the present description therefore has no added water, which is completely replaced by the tea infusion.

In possible embodiments, the cosmetic composition is an emulsion, in particular an emulsion cream. In this case, the tea infusion according to the present description completely replaces the emulsion water normally used.

The tea infusion according to embodiments described here is used as it is, without being subjected to other working or treatment, fermentation or other.

In possible variant embodiments, combinable with all the embodiments described here, it is possible to use infusions of different varieties of tea, such as green tea (for example Matcha), white tea, blue tea (for example Oolong), black tea (for example Assam, Darjeeling, Ceylon), red tea, or combinations or mixtures thereof. Typically, as we said, the tea is supplied in leaves.

In particular, embodiments can provide one or the other of:
white tea: from plantations in east Asia (China), harvested manually from eighteen months to three years of maturity of the plant;
black tea: from plantations in south-east Asia (Sri Lanka, India), harvested manually from three to five years of maturity of the plant;
green tea: from plantations in east Asia (China), harvested manually from three to five years of maturity of the plant;
Oolong tea; plantations in East Asia (China) on mineral-rich ground;
red tea.

In some embodiments, the five varieties of tea described above are pure natural products, not subjected to fermentation treatment during working for preparing the infusion in question. The dried leaves therefore keep their original structure with their characteristic color and are rounded and folded over themselves.

For the purposes of the present description, the teas should be preserved in a cool and well-aired place, in a well-closed container, away from humidity and heat.

According to the present description, the tea infusion is prepared using water heated to a suitable temperature, to which the tea leaves are added. After a certain infusion time, possibly preceded and/or followed by mixing, the infusion is discharged and filtered. The infusion technique used includes strategies whereby the tea leaves are not subjected to excessive heat stress, so as to preserve the beneficial properties of the compounds originally contained in the tea leaves.

According to possible embodiments, combinable with all the embodiments described here, in the method for preparing the tea infusion, the ratio of water(ml)/tea(grams) goes from 10:0.2 to 10:0.1. For example, the ratio can be 10:0.2, 10:0.15, 10:0.1. An example in the case of black tea can be from 1 to 1.5 grams of tea for 100 ml of water. Another example in the case of white tea can be 2 grams of tea for 100 ml of water. Another example in the case of green tea can be 1 gram of tea for 100 ml of water. Another example in the case of blue tea can be from 1 to 1.5 grams of tea for 100 ml of water. Another example in the case of red tea can be from 1 to 1.5 grams of tea for 100 ml of water. It is understood that 1 ml of water corresponds to 1 gram of water.

According to some possible embodiments, combinable with all the embodiments described here, the water used can be demineralized water which, for example, can come from an inverse osmosis demineralization plant. Advantageously, the inverse osmosis plant is able to guarantee a purity of the demineralized water of pharmaceutical quality, that is, a demineralized water is used with characteristics suitable for pharmaceutical production, which is known to have stricter purity parameters. This allows to have a high guarantee of drastically reducing the pollutants in the water produced. The demineralized water is also advantageously disinfected.

According to some embodiments, for preparing the tea infusion in question, the method provides that the water is weighed and loaded into a suitable container provided with a closing lid with a loading aperture (like a porthole), a heating/cooling system, for example using a heat exchange jacket, and mixing members, for example the planet type. A container of this type can be for example a turbo-emulsifier, to which we will refer hereafter for the purposes of the present description, but this shall not necessarily constitute a limit to the field of the present invention. The water can advantageously be loaded by vacuum suction, activating a suction pump (airless function).

After it has been loaded into the turbo-emulsifier, which remains closed, the water is taken to boiling inside the turbo-emulsifier. The temperature of the water can be controlled by a temperature probe which can be incorporated with the turbo-emulsifier. The boiling temperature can be that of water at ambient pressure, although working in a vacuum is not excluded, to reduce the boiling temperature. This last solution can contribute to preserving the natural properties of the more thermolabile components. When the required temperature is reached, the heating system of the turbo-emulsifier is switched off. Switching off the heating system causes the temperature of the water present therein to begin to go down. Then, through the loading aperture of the turbo-emulsifier the tea leaves are added inside, in the desired quantities depending on the quantity of water loaded, according to the ratios indicated above. The tea leaves are loaded, as we said, when the water in the turbo-emulsifier, after the heating system has been switched off, has cooled and reached a desired temperature correlated to the variety of tea used on each occasion. For example, the tea leaves are loaded when the water in the turbo-emulsifier has reached a temperature between 60° and 95°, depending on the variety of tea.

After the tea leaves have been added to the turbo-emulsifier, mixing can be started, for example by activating the planet mixer. The mixing can last for a time sufficient to guarantee homogeneous contact between the tea leaves and the hot water. Once mixing is switched off, with the turbo-emulsifier closed, the tea leaves are left to infuse, that is soaking in the hot water for from 30 seconds to 180 seconds, in particular from 45 seconds to 120 seconds, more particularly from 50 seconds to 90 seconds, and even more particularly from 60 seconds to 70 seconds. For example, a possible infusion time can be 60 seconds (1 minute). The infusion time selected can depend on the type of tea used. The infusion time is useful because, during this period, the tissues of the tea leaves open and stretch completely, allowing the hot water to extract the hydrosoluble active principles from it without altering the chemical structure and guaranteeing a high maintenance of the original properties of the tea. This extraction technique with the heating system switched off and the mixing switched off, that is, with the turbo-emulsifier not active, is advantageously intended to extract the components which otherwise would deteriorate during boiling due to heat stress. For example, this is advantageous to proceed safely with the extraction of the catechins present in the tea leaves, catechins which would be thermolabile at the boiling temperature of water. Catechins are hydrosoluble compounds that are effectively extracted in the tea infusion, but they can be affected by high process temperatures. Thanks to the extraction and infusion operations described above, where the temperature is properly controlled, the catechins, like the other beneficial compounds naturally present in tea leaves, are not affected by the temperature during their extraction and are therefore maintained in the final tea infusion, contributing the beneficial functions for which they are sought, for example maintaining their anti-oxidant capacities.

When infusion time is finished, cooling is performed by means of a rapid reduction in the temperature, activating the cooling of the turbo-emulsifier, taking the temperature to about 15-25° C., in particular 18-22° C., for example about 20° C. This rapid reduction in temperature has the advantageous effect of preserving essentially unchanged all the natural properties of the tea infusion produced and of the beneficial compounds and active principles present therein, such as polyphenols (catechins), vitamins, minerals, useful for the health of the organism, for example the skin, nails, hair, etc.

After cooling by rapid temperature reduction, another mixing can be provided, for example for 5 to 20 seconds, in particular from 7 to 15 seconds, for example 10 seconds.

Subsequently, the tea infusion produced is discharged and filtered, for example using a 100% polypropylene filter of 175 g/m². The tea infusion can be discharged into suitable food-quality sterile bags, which are in turn disposed in kegs or containers.

In possible embodiments, the tea infusion is produced as needed when there is a production need, and the whole quantity produced is used without adding preservatives or other additives.

The tea leaves used to make the tea infusion as described here typically contain, in a native and natural way, beneficial compounds such as polyphenols (catechins), purine bases (caffeine or theine), flavonoids, tannins, anti-oxidants, vitamins and minerals in general. The tea infusion according to the present description can be used for example in the formula of anti-ageing cosmetics and products able to control skin damage caused by exposure to environmental pollutants and cosmetics useful in the treatment of skin blemishes. The tea infusion described here can have the following cosmetic functions: anti-oxidant (anti-radical), astringent, skin purifier, eudermic, lipolytic (treatment of localized adiposity), sebum-equilibrium, tonic and stimulant.

Advantageously, the tea infusion in the cosmetic composition according to the present description functions as a natural source of said beneficial compounds, including polyphenols, EGCG catechins (epigallocatechin gallate), anti-oxidants, caffeine, theobromine, tannins, vitamins and minerals, which are normally contained in tea leaves and released during infusion. These beneficial compounds promote the recovery of properties and characteristics of the skin. These beneficial compounds, in particular polyphenols, anti-oxidants, vitamins and minerals, can also have bactericide and/or bacteriostatic properties, and can contribute to the preservation of the cosmetic composition itself, reducing or eliminating the need to use other preservatives.

Advantageously, thanks to the technique of preparing the tea infusion described here, which does not subject to heat stress the beneficial compounds present in the tea leaves, such as polyphenols (catechins), the beneficial compounds can also be found in the tea infusion obtained, and the beneficial effects, for example anti-oxidant, of such compounds in the tea leaves is also preserved in the tea infusion, as shown by the experimental tests carried out by Applicant and described hereafter.

In some embodiments, combinable with all the embodiments described here, the cosmetic composition can include tea infusion in a concentration greater than 50% w/w, preferably comprised between 55% and 95%. For example, the tea infusion can be present in the cosmetic composition in question from 50% to 60% w/w, or from 55% to 65% w/w, or from 60% w/w to 70% w/w, or again from 65% w/w to 75% w/w, or from 70% w/w to 80% w/w.

In some embodiments, combinable with all the embodiments described here, the tea infusion can possibly be associated with one or more preservatives. Examples of possible preservatives used are sodium dehydroacetate, for example from 0.01% to 0.3% w/w, sodium benzoate, for example from 0.01% to 0.1% w/w, potassium sorbate for example from 0.01% to 0.1% w/w.

According to some embodiments, combinable with all the embodiments described here, the at least one composition component for personal care present in the cosmetic composition can preferably be a surfactant, chosen from an anionic surfactant, a zwitterionic surfactant, a cationic surfactant, a non-ionic surfactant, present in the cosmetic composition in a concentration generally comprised between 0.5 and 16.5% w/w, preferably between 1 and 5% w/w.

Furthermore, the component for personal care can be chosen from a group consisting of: a lipid, an alcohol, a fatty alcohol, a fatty alcohol of vegetable origin, an ester, a natural ester, a wax, a pigment, a vitamin, a fragrance, a whitening agent, an antibacterial agent, an anti-inflammation agent, an anti-mycotic agent, a thickener, a starch, chitosan, a polymer material, a cellulose material, glycerin, a protein, an amino acid, a keratin fiber, a fatty acid, a siloxane, an abrasive, an exfoliator, lanolin, an anti-caking agent, an anti-oxidant agent, a binder, a biological additive, a buffering agent, a bulking agent, a chelant, a chemical additive, a denaturant, an external analgesic, a filming agent, a humectant, an opacifier agent, a pH regulator, a preservative, a propellant, a reducing agent, a solar screen agent, a skin darkening agent, an essential oil, an agent for sensitive skin.

In some embodiments, combinable with all the embodiments described here, the cosmetic composition in emulsion form can be multi-phase, for example bi-phase, tri-phase, four-phase, five-phase or even more.

In some embodiments, combinable with all the embodiments described here, the cosmetic composition comprises a liquid lipophilic phase, or oil phase, and the tea infusion, to completely replace the aqueous phase normally present in emulsion creams.

In some embodiments, combinable with all the embodiments described here, the oil phase can be present in the cosmetic composition from 15% to 25% w/w, in particular from 16% to 22% w/w, more particularly from 18% to 20% w/w.

In some embodiments, combinable with all the embodiments described here, the oil phase can include one or more vegetable oil compounds. The vegetable oils are typically in a liquid state at ambient temperature. In particular, the oil phase can include one or more or all the following oil compounds or combinations thereof: Limnanthes alba oil, rice oil, grapeseed oil, jojoba oil, babassu oil, argan oil, almond oil, castor oil, coconut oil, maize oil, cottonseed oil, rapeseed oil, linseed oil, hempseed oil, nut oil, palm oil, hazelnut oil, safflower oil, sesame oil, soya oil, sunflower oil.

The oil compounds described above can be used all together, or only part or some of them, choosing each time the corresponding compositions according to the intervals indicated above.

In possible implementations, the oil phase can include one or more preservatives, for example phenoxyethanol, which can be from 0% to 1% w/w.

In some embodiments, combinable with all the embodiments described here, the cosmetic composition comprises the liquid lipophilic phase, or oil phase, the tea infusion and a wax phase.

In some embodiments, combinable with all the embodiments described here, the wax phase can be present in the cosmetic composition from 15% to 25% w/w, in particular from 16% to 22% w/w, more particularly from 18% to 20% w/w.

In some embodiments, combinable with all the embodiments described here, the wax phase can include one or more wax compounds, in particular one or more or all wax compounds, wax emulsifiers, emulsifying wax esters. The wax compounds are typically in a solid state at ambient temperature, and have a waxy consistency.

In some embodiments, combinable with all the embodiments described here, the cosmetic composition comprises the lipophilic phase, the tea infusion, the possible wax phase, a phase of active and/or synergizing compounds and/or a fragrance phase and/or one or more colorants.

In some embodiments, combinable with all the embodiments described here, the phase of active and/or synergizing compounds can be present in the cosmetic composition from 2% to 15% w/w, in particular from 3% to 12% w/w, more particularly from 5% to 10% w/w.

In some embodiments, combinable with all the embodiments described here, the phase of active and/or synergizing compounds can include one or more of the following active and/or synergizing compounds: vitis vinifera seed oil, boswellia serrata extract, royal jelly extract, extract of oats, extract of millet, dog rose extract, fenugreek extract, centella extract, ivy extract, horse chestnut extract, sodium hyaluronate, dimethylsilanol hyaluronate, beta glucan, coenzyme Q10.

The active and/or synergizing compounds described above can be used all together, or only part or some of them, or even only one of them, choosing each time the corresponding compositions according to the intervals indicated above. For example, specific cosmetic compositions can be obtained using a specific one or a group of selected specific active and/or synergizing compounds described above.

In some embodiments, combinable with all the embodiments described here, the fragrance phase goes from 0% to 0.2% w/w.

In some embodiments, combinable with all the embodiments described here, the cosmetic composition can be completely without compounds or ingredients deriving from fossil fuels, in particular petrochemicals, that is, compounds deriving from petroleum (petrolatum).

In some embodiments, combinable with all the embodiments described here, the cosmetic composition can be completely without ingredients or materials of animal or vegetable origin which are obtained by using genetically modified organisms.

In some embodiments, combinable with all the embodiments described here, the cosmetic composition can be a cream, in particular a bi-phase cream, comprising said oil phase and the tea infusion, or tri-phase, comprising oil phase, wax phase and tea infusion, or multi-phase, in the case of added phase of active and/or synergizing compounds and/or fragrance phase. In possible implementations, the cream can be, in particular, an oil emulsion, or oil and wax, in the tea infusion, or tea infusion in oil, or oil and wax, depending on which of the two components or phases is dispersed (dispersed or discontinuous phase) in the other (carrier or continuous phase).

In some embodiments, the cosmetic composition can be a cream for skin care, a body cream, a face cream, a hand cream, a leg cream, a foot cream, a hair cream, a soothing balsam cream for the hair, a cream for around the eyes or for the eyelids, or an anti-cellulite, slimming or remodeling cream or gel, an exfoliator product for the face or body, a make-up removing lotion, UVA-UVB protection creams or lotions, tanning creams and lotions, perfumes for the body, make-up products such as foundation cream, powder or compacts, lipsticks or lip balms, tanning powders.

In a preferred embodiment of the present invention, the cosmetic composition is a cream lotion for personal care comprising:
an infusion of tea in a concentration comprised between 55% and 90% w/w, preferably between 60% and 80% w/w;
an oil phase in a concentration comprised between 5% and 25% w/w, preferably between 10% and 22% w/w;
a wax phase in a concentration comprised between 2% and 24% w/w, preferably between 10% and 20% w/w;
active and/or synergizing compounds in a concentration comprised between 0.5% and 15% w/w, preferably between 2% and 10% w/w;
excipient compounds in a concentration comprised between 1% and 15% w/w, preferably between 2% and 10% w/w;
surfactants in a concentration comprised between 0.5% and 16.5% w/w, preferably between 1% and 5% w/w.

In preparing the cream lotion for personal care as above, an infusion of green tea is preferably used.

As components of the oil phase, ethylhexyl palmitate, decyl oleate and dimethicone can be cited for example.

As components of the wax phase, butyrosspermum parkii butter, stearyl alcohol, glyceril stearate and cetearyl glucoside can be cited for example.

As active and/or synergizing compounds tocopheryl acetate, persea gratissima butter, sodium hyaluronate and triticum vulgare germ oil can be cited for example.

As excipient compounds, propanediol, glycerin, phenoxyethanol and ethylhexylglycerin can be cited for example.

In another preferred embodiment of the invention, the cosmetic composition is a serum cream for the face, comprising:
infusion of tea in a concentration comprised between 60% and 95% w/w, preferably between 70% and 92% w/w;
active and/or synergizing compounds in a concentration comprised between 0.5% and 15% w/w, preferably between 2% and 10% w/w;
excipient compounds in a concentration comprised between 1% and 15% w/w, preferably between 2% and 10% w/w;
surfactants in a concentration comprised between 0.5% and 16.5% w/w, preferably between 1% and 5% w/w.

In preparing the cream serum for the face as above, an infusion of white tea is preferably used.

As surfactant agents PEG-40 hydrogenated castor oil, PEG-6 caprylic/capric glycerides can be cited for example.

As active and/or synergizing compounds tocopheryl acetate, ascorbyl tetraisopalmitate, ascorbic acid and olea europaea leaf extract can be cited for example.

As excipient compounds propanediol, glycerin, phenoxyethanol and ethylhexylglycerin can be cited for example.

Embodiments described here concern a method for preparing a cosmetic composition without an added aqueous phase according to the present description, the method including:
preparing the tea infusion according to embodiments described here;
combining the tea infusion with at least one composition component for personal care.

If the cosmetic composition is an emulsion, the tea infusion completely replaces the aqueous phase normally used in emulsions, and an emulsion is prepared of the tea infusion and an oil phase.

In possible embodiments, combinable with all the embodiments described here, the preparation of the cosmetic composition in an emulsion with no added aqueous phase provides to prepare the oil phase by assembling the oil compounds cold.

If the cosmetic composition also includes a wax phase, the method also can provide to prepare the wax phase separately, assembling the wax compounds hot, in particular taking them to melting point. Once the oil compounds have been assembled cold, the oil phase thus prepared can be added to the possible wax phase made separately.

Subsequently, the oil phase and the possible wax phase if present can be added, that is dispersed, in the tea infusion, previously prepared and cooled as described above, obtaining the emulsion. Furthermore, if provided, it is possible to add the phase of active and/or synergizing compounds and/or the fragrance phase.

Consequently, embodiments described here, unlike in the state of the art, can provide to prepare the oil phase cold, and possibly to prepare hot by melting the possible wax phase if present, and subsequently to add them to the tea infusion, prepared and cooled, which replaces the aqueous phase, in which they are dispersed.

According to possible implementations, it is possible to use a turbo-emulsifier to prepare the cosmetic composition, that is, a mixer provided with a stirrer, which can typically be a planet mixer, for example with anchors and blades, and with a turbine and a suction pump to obtain a vacuum state (airless function), and a fuser, provided with a heating sleeve and connected to the turbo-emulsifier by means of a pipe. The passage of fluid through the pipe is allowed, managed and controlled by a valve, for example a tap, associated with the pipe, generally in the lower part of the turbo-emulsifier. The pipe can be provided with a filter. The oil phase is loaded into the fuser. If provided in the formula, the wax phase is melted in the fuser, for example at a temperature of about 70° C.-80° C., and the oil phase described above is added to it. Simultaneously, the tea infusion is loaded in a turbo-emulsifier. The tea infusion can be heated to about 35° C.-45° C. in the turbo-emulsifier, or used at ambient temperature.

Subsequently, when the oil phase with the possible wax phase in the fuser, and the tea infusion in the turbo-emulsifier are all ready, a vacuum is activated in the turbo-emulsifier and, opening the valve in the pipe that connects fuser and turbo-emulsifier, the oil phase and the possible wax phase are sucked up by the pump with the turbine activated in the turbo-emulsifier. The turbine advantageously refines and homogenizes the particles finely. In this way the oil phase and the possible wax phase are emulsified with the tea infusion. The emulsification can proceed for a desired time, from a few seconds to a few tens of seconds, and as long as some minutes or tens of minutes, for example even 30 minutes depending on the type of formula, the turbo-emulsifier and the quantity to be produced. After which, when the emulsion is incipient, and at a temperature of about 25° C.-30° C., the phase of active and/or synergizing compounds can be added, if provided in the formula. After this the process continues with emulsification and homogenization for a few minutes and finally the process can possibly be completed with the addition of the fragrance phase. The vacuum is activated to free the emulsion of air incorporated in the mixing and subsequent homogenization, to prevent the semi-worked product from appearing “swollen” and hence not very pleasant. After further emulsification and homogenization through stirring, for example lasting from 8 to 12 minutes, in particular from 9 to 11 minutes, the product can be transferred to suitable containers and is left to rest, for example from 10 to 14 hours, in particular from 11 to 13 hours. During the resting phase chemical-physical checks can be made on the product, such as checks on the pH, viscosity, specific weight, and microbiological checks, such as a check on the microbe load, search for pathogens if necessary.

### EXPERIMENTAL TESTS

Applicant has carried out experimental tests to verify and demonstrate that the anti-oxidant capacity of the beneficial compounds naturally present in tea leaves which are extracted in the tea infusion according to the present description is suitably preserved even after the tea infusion has been stored and is found satisfactorily even in the final cosmetic compositions produced.

### Samples Analyzed

To this purpose, Applicant compared the anti-oxidant capacity of three extemporaneous infusions of tea (i, ii and iii), that is, produced on the moment for the purposes of the analysis, a tea infusion (iv) produced previously and stored as described above (1% infusion), and two cosmetic products, that is, a cream for around the eyes (v) and a serum in drops (vi), both formulated by Applicant containing the tea infusion according to the present description:

i) extemporaneous infusion of black tea leaves;
ii) extemporaneous infusion of green tea leaves;
iii) extemporaneous infusion of white tea leaves;
iv) infusion of white tea leaves (1%);
v) cream for around the eyes containing an infusion of white tea;
vi) serum in drops containing an infusion of white tea.

### Method of Analysis

The analysis was carried out using a photo chemiluminescence (PCL) method, to assess the anti-oxidant capacity of the above samples. The PCL method allows to quantify both the anti-oxidant capacity of pure hydrophilic and/or lipophilic substances, and also to measure the anti-oxidant capacity in the lipidic phase (ACL) and in the aqueous phase (ACW) of complex matrixes of various origin. With this method, the anti-oxidant properties of the substances examined can be determined quickly, reliably and effectively, limiting the losses of activities connected to degradation. The method is based on the photo-induced oxidation of luminol, mediated by the radical superoxide anion (O^{2.-}) generated photochemically and accompanied by intense chemiluminescence. The presence of anti-oxidants inhibits the auto-oxidation of the luminol, which assumes the role both of photosensitizer and also of chemiluminescent reagent. In the presence of substances that act as traps for radicals (anti-oxidants), the intensity of the PCL is attenuated as a function of their activity and concentration. The test can be carried out according to two different protocols, ACW and ACL, which allow to show the anti-oxidant capacity of the hydrosoluble and liposoluble component respectively. In fact, anti-oxidants belonging to the flavonoid family normally come within the hydrosoluble component, whereas compounds such as tocopherol, tocotrienol, carotenoids etc. come within the liposolubles. The ACL protocol uses Trolox® as reference standard, while ascorbic acid is used for ACW: the results are therefore expressed respectively in equivalents of Trolox® and equivalents of ascorbic acid. The IAC (Integral Antioxidant Capacity), expressed in equivalents of Trolox® per sample unit, represents the total anti-oxidant capacity obtained with the ACQ and ACL protocols.

### Results

The Table below shows the values of anti-oxidant capacity of the samples analyzed, obtained for each test from three independent analyses.

| | PCL Test - ACL micromoles |
|---|---|
| | Trolox ®/grams (P ≤ 0.05) |
| i) extemporaneous infusion of black tea | 8.8 ± 0.00 |
| leaves | |
| ii) extemporaneous infusion of green tea | 4.13 ± 0.07 |
| leaves | |
| iii) extemporaneous infusion of white tea | 3.25 ± 0.07 |
| leaves | |
| iv) infusion of white tea leaves (1%) | 3.24 ± 0.03 |
| v) cream for around the eyes containing an | 1.40 ± 0.01 |
| infusion of white tea | |
| vi) serum in drops containing an | 2.12 ± 0.07 |
| infusion | |
| of white tea | |

From the analyses carried out, sample i) showed the highest anti-oxidant activity, while the other two samples ii) and iii) showed comparable activity. Sample iv) and sample iii) gave similar results in terms of anti-oxidant activity to prove and confirm that, thanks to the preparation technique described above, the tea infusion maintains the anti-oxidant capacities even after preparation. Furthermore, this confirms the expected anti-oxidant capacity of the samples of cosmetic composition v) and vi), as a function of the quantity of tea infusion present in them.

The following Table shows the anti-oxidant capacity of some reference products with anti-oxidant activity, with which to compare the results obtained.

| | PCL Test - ACL micromoles |
|---|---|
| | Trolox ®/grams |
| Emulsion with ascorbic acid 0.25% | 21.9 |
| w/w | |
| Emulsion with ascorbic acid 0.5% | 39.7 |
| w/w | |
| Emulsion with tocopherol 2.5% | 84.7 |
| w/w | |
| Ascorbic acid | 16120 |
| α-tocopherol | 2770 |

From this comparison, it is obvious that the tea infusion and the products and cosmetic compositions containing the tea infusion described and tested here maintain an adequate and satisfactory anti-oxidant capacity, compared with compounds known for their native anti-oxidant properties.

### EXAMPLES OF COSMETIC COMPOSITION

### Example 1

There follows an example of a cosmetic composition according to the invention, that is, a cream lotion for body care, which is totally without added aqueous phase and comprises an infusion of green tea to replace the aqueous phase.

The cream lotion for body care has the following composition:
concentration of infusion of green tea: 65% w/w;
concentration of oil phase: 11.8% w/w;
concentration of wax phase: 10% w/w;
concentration of active and/or synergizing compounds: 1% w/w;
concentration of fragrance phase: 2% w/w;
concentration of excipients: 8.45% w/w;
concentration of surfactants: 16.5% w/w.

### Example 2

There follows an example of a cosmetic composition according to the present invention, that is, a serum cream for the face, which is totally without added aqueous phase and comprises an infusion of white tea to replace the aqueous phase.

The serum cream for the face has the following composition:
concentration of infusion of white tea: 91.67% w/w;
concentration of active and/or synergizing compounds: 0.25% w/w;
concentration of excipients: 6.18% w/w;
concentration of surfactants: 1.9% w/w.

It is clear that modifications and/or additions of parts may be made to the cosmetic composition as described heretofore, without departing from the field and scope of the present invention.

It is also clear that, although the present invention has been described with reference to some specific examples, a person of skill in the art shall certainly be able to achieve many other equivalent forms of cosmetic composition, having the characteristics as set forth in the claims and hence all coming within the field of protection defined thereby.

1. Cosmetic composition, with no added aqueous phase and comprising a tea infusion to replace the aqueous phase, combined with at least one composition component for personal care,
wherein said tea infusion is present in a concentration between 55% and 95%, and is an infusion of one or more teas chosen from a group consisting of: white tea, black tea, green tea, blue tea and red tea, and
wherein said cosmetic composition is a cream comprising:
an infusion of tea in a concentration between 55% and 90% w/w;
an oil phase in a concentration between 5% and 25% w/w;
a wax phase in a concentration between 2% and 24% w/w;
active and/or synergizing compounds in a concentration between 0.5% and 15% w/w;
excipient compounds in a concentration between 1% and 15% w/w; and
surfactants in a concentration between 0.5% and 16.5% w/w.

2. Cosmetic composition as in claim 1, wherein said composition component for personal care is a surfactant chosen from an anionic surfactant, a zwitterionic surfactant, a cationic surfactant and a non-ionic surfactant.

3. Cosmetic composition as in claim 1, wherein said cosmetic composition is for topical application on the skin, and/or hair and/or nails.

4. Cosmetic composition as in claim 1, wherein said cream comprises:
an infusion of tea in a concentration between 60% and 80% w/w;
an oil phase in a concentration between 10% and 22% w/w;
a wax phase in a concentration between 10% and 20% w/w;
active and/or synergizing compounds in a concentration between 2% and 10% w/w;
excipient compounds in a concentration between 2% and 10% w/w;
surfactants in a concentration between 1% and 5% w/w.

5. Cosmetic composition, with no added aqueous phase and comprising a tea infusion to replace the aqueous phase, combined with at least one composition component for personal care,
wherein said tea infusion is present in a concentration between 55% and 95%, and is an infusion of one or more teas chosen from a group consisting of: white tea, black tea, green tea, blue tea and red tea,
wherein said cosmetic composition is a cream for the face comprising:
an infusion of tea in a concentration between 55% and 95% w/w;
active and/or synergizing compounds in a concentration between 2% and 10% w/w;
excipient compounds in a concentration between 1% and 15% w/w; and
surfactants in a concentration between 0.5% and 16.5% w/w.

6. Cosmetic composition as in claim 5, wherein said cream comprises:
an infusion of tea in a concentration between 70% and 92% w/w;
active and/or synergizing compounds in a concentration between 2% and 10% w/w;
excipient compounds in a concentration between 2% and 10% w/w; and
surfactants in a concentration between 1% and 5% w/w.

7. Method for preparing a tea infusion for use in the preparation of a cosmetic composition, with no added aqueous phase and comprising said tea infusion to replace the aqueous phase, combined with at least a composition component for personal care, said method comprising:
weighing the water and loading it by means of vacuum suction into a turbo-emulsifier and bringing it to the boil by activating a heating system of the turbo-emulsifier;
switching off the heating system to make the temperature of the water go down to a temperature correlated to the type of tea leaves used for the infusion;
adding the tea leaves to the water in the turbo-emulsifier;
mixing the tea leaves and water in the turbo-emulsifier;
stopping the mixing;
letting the infusion of tea leaves continue in the hot water in the turbo-emulsifier for a predefined infusion time;
cooling the tea infusion obtained by rapidly bringing down the temperature in the turbo-emulsifier;
filtering and packing the tea infusion.

8. Method as in claim 7, wherein the addition of tea leaves is made when the water in the turbo-emulsifier has reached a temperature between 60° C. and 95° C.

9. Method as in claim 7, wherein the infusion time is from 30 seconds to 180 seconds, in particular from 45 seconds to 120 seconds, more particularly from 50 seconds to 90 seconds, even more particularly from 60 seconds to 70 seconds.

10. Method as in claim 7, wherein the rapid bringing down of the temperature takes the tea infusion to a temperature of about 15-25° C., in particular 18-22° C.

11. Method to prepare a cosmetic composition in emulsion and with no added aqueous phase, said method providing to use a tea infusion to replace the aqueous phase prepared as in a method as in claim 7 and the combination of the tea infusion with at least a composition component for personal care.

12. Cosmetic composition as in claim 1, wherein said cream is a body cream.

13. Cosmetic composition as in claim 5, wherein said cream is a serum cream.
